(19) United States
(12) Patent Application Publication
(10) Pub. No.: US 2008/0026411 A1
(43) Pub. Date: Jan. 31, 2008

(54) Diagnostic and prognostic methods and compositions of matter for cell proliferative diseases

(76) Inventors: Jiangping Wu, Brossard (CA); Yulian Wu, Hangzhou (CN)

Correspondence Address: GOUDREAU GAGE DUBUC, 2000 MCGILL COLLEGE, SUITE 2200, MONTREAL, QC, H3A 3H3, US

(21) Appl. No.: 10/933,244

(22) Filed: Sep. 3, 2004

Related U.S. Application Data
(60) Provisional application No. 60/499,732, filed on Sep. 4, 2003.

(51) Int. Cl. G01N 33/53

(52) U.S. Cl. 435/7.92; 435/40.51; 436/86

(57) ABSTRACT

A method for diagnosing a cell proliferative disease expressing DcR3/TR6 in a patient, which comprises the step of measuring the concentration of DcR3/TR6 in the patient's blood, plasma or serum sample, wherein a concentration of DcR3/TR6 higher than that present in the serum of a patient not suffering of a proliferative disease expressing DcR3/TR6 is indicative of that patient suffering from said disease. Methods of prognosing said diseases and compositions of matters for use in said diseases.

### FIELD OF THE INVENTION

The present invention relates to a method and a composition of matter for diagnosing a cell proliferative disease such as cancer and liver cirrhosis from blood samples. More specifically, the present invention is concerned with detecting DcR3 levels in blood samples.

### BACKGROUND OF THE INVENTION

TNF family receptor 6 (TR6), also known as decoy receptor 3 (DcR3) or M68, is a new member of the TNF receptor family (TNFR) (1). It lacks an apparent transmembrane domain in its sequence, and is a secreted protein (1,2). It can bind to a TNF family member, FasL, and prevent FasL-induced apoptosis (1). TR6 also binds to LIGHT, which is another member of the TNF family (3) and is expressed on activated T-cells (4) and immature dendritic cells (5). LIGHT is found to induce apoptosis in cells expressing both HveA/TR2 and LTβR (6), or LTβR alone (7), both of which are receptors of LIGHT. TR6 can thus also prevent LIGHT-triggered apoptosis (3). It interacts with a third ligand, TL1A (8), a new member of the TNF family. DR3, a death receptor belonging to the TNFR family, is the bona fide cell surface receptor of TL1A (8). Therefore, one of the physiological functions of TR6 is to act as a death decoy to prevent apoptosis mediated by TNFR family members such as Fas, LTβR and DR3. Since LIGHT and HveA/TR2 bind to each other and transduce costimulatory signals bidirectionally into T cells (2,9,10), the second physiological function of TR6 is to repress T-cell activation by blocking the bidirectional costimulation between HveA/TR2 and LIGHT on T cells. Through a so-far uncharacterised mechanism, TR6 reportedly modulates the function of dendritic cells, which, in turn, deviate T-cell responses towards the Th2 phenotype (11).

An initial report on TR6 has indicated that TR6 is expressed at high levels in some gastrointestinal tumours (1). Conceivably, TR6 secreted by these tumours might protect them from apoptosis induced by FasL, LIGHT, and/or TL1A; it might also suppress or deviate immune surveillance by blocking the T-cell costimulation mediated by TR2 and LIGHT, and by modulating dendritic cell function. Consequently, tumours secreting TR6 gain a survival advantage.

Since TR6 is a protein preferentially secreted by proliferating cells, it would be of great interest to determine whether there is any indication or correlation between blood, plasma or serum sample TR6 levels and the presence or stage of a proliferative disease. A reliable blood, plasma or serum sample marker for certain types of cancer or other proliferative diseases would provide a convenient diagnostic and prognostic tool, non-invasive and measurable simply with a patient's blood sample. The present invention seeks to meet these needs and other needs.

The present description refers to a number of documents, the content of which is herein incorporated by reference in their entirety.

### SUMMARY OF THE INVENTION

This invention correlates serum TR6 levels to various cancers and cancer stages.

More specifically, in accordance with the present invention, there is provided a method for diagnosing a cell proliferative disease expressing DcR3/TR6 in a patient, which comprises the step of measuring the concentration of DcR3/TR6 in the patient's blood, plasma or serum sample, wherein a concentration of DcR3/TR6 higher than that present in the serum of a patient not suffering of a proliferative disease expressing DcR3/TR6 is indicative of that patient suffering from said disease. In a more specific embodiment, the patient's sample comprises at least about 20 pg/mL DcR3/TR6. In a further more specific embodiment, said disease is selected from the group consisting of gastric, liver, pancreatic, gallblader, colon, thyroid, lung, bone, larynx or breast cancers, and liver cirrhosis. In a further more specific embodiment, the measuring step is selected from the group consisting of ELISA, radioimmunoassay, flow cytometry, fluorometry and immunoblotting. In a further more specific embodiment, the detecting step comprises the use of an anti-DcR3/TR6 antibody. In a further more specific embodiment, said patient is a patient who has undergone tumor resection at least about 4 weeks before said step of measuring DcR3/TR6 and wherein a concentration of DcR3/TR6 equal or lower than that present in the sample of a patient not suffering of a proliferative disease expressing DcR3/TR6 is indicative of the success of the tumor resection. In a further more specific embodiment, said patient is one who has undergone tumor resection at least about 4 weeks before said step of measuring DcR3/TR6, and wherein a concentration of DcR3/TR6 higher than that present in the sample of a patient not suffering of a proliferative disease expressing DcR3/TR6 is indicative of a tumor recurrence. In a further more specific embodiment, said patient is one who has undergone tumor resection at least about 4 weeks before said step of measuring DcR3/TR6 and whereby the tumor recurrence after tumor resection can be monitored. In a further more specific embodiment, a concentration of DcR3/TR6 higher than that present in the sample of a patient not suffering from a proliferative disease expressing DcR3/TR6 is further indicative of the presence of metastasis or tumor invasion in the patient. In a further more specific embodiment, the patient sample is serum.

In accordance with an other aspect of the present invention, there is provided a method for predicting whether a patient suffering from a cell proliferative disease is at risk of experiencing a disease progression or recurrence which comprises the step of measuring the concentration of DcR3/TR6 in the patient's blood, plasma or serum sample, wherein said sample DcR3/TR6 concentration positively correlates with the disease clinical stage. In a more specific embodiment, said patient's sample comprises at least about 20 pg/mL DcR3/TR6. In a more specific embodiment, said disease is selected from the group consisting of gastric, liver, pancreatic, gallblader, colon, thyroid, lung, bone, larynx or breast cancers, and liver cirrhosis. In a further more specific embodiment, said disease is gastric cancer. In a further more specific embodiment, the measuring step is selected from the group consisting of ELISA, radioimmunoassay, flow cytometry, fluorometry and immunoblotting. In a further more specific embodiment, the detecting step comprises the use of an anti-DcR3/TR6 antibody. In a further more specific embodiment, the patient sample is serum.

In a further aspect of the present invention, there is provided a composition of matter for the quantitative detection of DcR3/TR6 in a patient's blood, plasma or serum sample, which comprises a ligand to DcR3/TR6, reactants supporting the formation of a complex between said ligand and DcR3/TR6 and the detection of said complex, and a predetermined amount of DcR3/TR6 to be submitted to serial dilutions and to be mixed with a control sample, providing a DcR3/TR6 standard curve. In a more specific embodiment, said ligand is an anti-DcR3/TR6 antibody. In a further more specific embodiment, said DcR3/TR6 serial dilutions achieve a concentration range expanding from 0.1 pg/mL to 1000 pg/mL sample.

In a further aspect of the present invention, there is provided a composition of matter for predicting a risk incurred by a patient suffering from a cell proliferative disease of experiencing a disease progression or recurrence, which comprises which comprises a ligand to DcR3/TR6, reactants supporting the formation of a complex between said ligand and DcR3/TR6 and the detection of said complex, and a predetermined amount of DcR3/TR6 to be submitted to serial dilutions and to be mixed with a control sample, providing a DcR3/TR6 standard curve.

Any method of measuring the concentration or level of the TR6 protein in a patient's blood, blood fraction or blood derived product such as plasma or serum may be used in accordance with the present invention according to methods known in the art. Such standard techniques can be found in relevant chapters of reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York) for instance. Without being so limited, these methods include ELISA, radioimmunoassay, flow cytometry, fluorometry and immunoblotting.

As used herein, the term “patient” refers to a mammal patient including a human patient that has a proliferative disease.

As used herein the term “cell proliferative disease” is meant to refer to a disease wherein the cells of a tissue or an organ has undergone an abnormal proliferation or hyperplasia, and may eventually become cancerous. Without being so limited, it refers to cancers such as gastric, liver, pancreatic, gallblader, colon, thyroid, lung, bone, larynx or breast cancers. Other cancers in addition to those listed herein wherein detection method could predictably work. Amongst non-cancer diseases which may become cancerous, liver cirrhosis is one such disease expressing high serum TR6 levels.

In another embodiment of the invention, specific binding molecules, such as antibodies or fragments thereof against a TR6 antigen, can be used to detect or image localization of the antigen in a patient for the purpose of detecting or diagnosing a proliferative disease or condition. Such antibodies can be polyclonal or monoclonal, or made by molecular biology techniques, and can be labeled with a variety of detectable labels, including but not limited to radioisotopes and paramagnetic metals.

In another embodiment of the invention, assay kit for determining the presence of TR6 antigen or anti-TR6 antibody in a test sample comprises a container containing a ligand or specific binding molecule which specifically binds to a TR6 antigen, such as an antibody or fragment thereof, a receptor or a fragment thereof, or an agonist or an antagonist molecule to TR6. The TR6 antigen comprises at least one TR6-encoded epitope. The TR6 antigen has at least about 50% sequence similarity to a sequence of a TR6-encoded antigen as set forth in SEQ ID NO: 1, and fragments thereof. Variants of this sequence are known and may be distinguished through the method of the present invention. These test kits can further comprise containers with components and reactants for receiving, processing, reacting the ligand with TR6 and detecting the reaction in the blood, plasma or serum samples. The kits may further comprise with tools useful for collecting test samples. Such tools include lancets and absorbent paper or cloth for collecting and stabilizing blood or a blood derived product. Collection materials, such as papers, cloths may optionally be treated to avoid denaturation or irreversible adsorption of the sample. These collection materials may also be treated with, or contain, preservatives, stabilizers or antimicrobial agents to help maintain the integrity of the specimens. The antibody or ligand can be attached to a solid phase. It could even be a cell or cell fraction capable of binding TR6 and providing a measurable signal upon said binding.

As used herein, the terminology “disease progression or recurrence” refers to the development or spread (in size or malignancy) of an existing tumor or appearance of new symptoms and tumors. Without being so limited, this terminology refers to lymph node metastasis, distant metastasis, biochemical recurrence and/or hormone refractoriness for instance.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 presents serum TR6 levels in tumor patients. Patient sera were collected before endoscopy or operation, and serum TR6 was assessed by ELISA in duplicated samples. The arbitrary TR6-positive threshold (solid line) was set at 20 pg/ml. Median TR6 levels are indicated by short bars. The number of patients tested (n) is shown;

FIG. 2 presents serum TR6 levels of patients with acute infection (cholecystitis or appendicitis), liver cirrhosis or liver carcinomas. Median TR6 levels are indicated by short bars. The number of patients tested (n) is shown.

FIG. 3 presents TR6 expression in tumor mass or liver cirrhosis according to IHC. Paraffin-embedded tumor sections were stained with affinity-purified rabbit anti-TR6 Ab, followed by Histostain-Plus™ staining system. TR6 signals are shown in brown, and are located in cytoplasma of cancer cells in the cancer cell nests. The liver cirrhosis sample was from the biopsy of a portal hypertension patient subjected to spleen resection. The original magnifications were 10× and 40×;

FIG. 4 presents serum TR6 levels after tumor resection. Sera of tumor patients were collected within 1 week before their curative operations and 4-6 weeks after their surgery;

FIG. 5 presents the relationship between serum TR6 levels and gastric carcinoma size. Serum TR6 levels (pg/ml) and gastric carcinoma sizes (cm) were plotted. The correlation index (r) was 0.022063, and was less than r _{(n′10, 0.05)}=0.671, hence p>0.05;

FIG. 6 presents serum TR6 levels of patients with tumors of different differentiation status and of patients with nonmalignant conditions. A. Serum TR6 levels of patients with highly or poorly differentiated gastric carcinomas. B. Serum TR6 levels of patients with thyroid adenomas and thyroid adenocarcinomas;

FIG. 7 presents the relative TR6 gene copy numbers in tumor mass. DNA was tested for TR6 and β-globin fluorescent signals (F1 and F2, respectively) by real time PCR. The means ±2SD (solid line and 2 dashed lines, respectively) of F1/F2 ratios of 6 normal blood DNA samples as indicated. The F1/F2 ratios representing 2 copies of TR6 gene with 99% confidence fall between the two dashed lines; and

FIG. 8 presents the amino acid (A) and nucleotidic (B) sequences (SEQ ID NO: 2 and 1, respectively) for TR3.

### DESCRIPTION OF ILLUSTRATIVE EMBODIMENTS

### Clinical Samples

Tumor patients' sera were from those undergone endoscopic biopsy, or diagnostic or curative operations. Sera from patients with nonmalignant conditions (acute appendititis, acute cholecystitis or liver cirrhosis) or from healthy individuals were collected during the same period of this project. Tumor DNA and tissues for immunohistochemisty were from fresh specimens of resection surgery. All samples were obtained with patient consent and local ethical committee approval.

### TR6 Elisa

Anti-TR6 mAb (clone 17B07) was previously described (2). The TR6 polyclonal antibody was purified from antisera generated from rabbits immunized with four synthetic peptides that spanned the TR6 protein sequence: V30-R46, R64-Q89, E240-R258, and R284-L297 (amino acid positions were relative to the start methionine). The nucleotide and amino acid sequences (SEQ ID NO: 2, and 1, respectively) of one TR6 can be found at accession number AF104419. Rabbit antisera were purified on a TR6-coupled Affi-Gel10™ column. The specificity of the TR6 polyclonal antibody was demonstrated in the ELISA by testing cross-reactivity to recombinant OPG and HveA, the two TNF receptor family members most closely related to TR6. Neither OPG nor HveA was detectable in the TR6 ELISA. The preparation of recombinant TR6 was described in detail previously (2). The protocol of TR6 ELISA is as follows. Ninety-six-well Nunc Maxisorb™ plates were coated overnight with anti-TR6 mAb in 0.05 M NaHCO₃ buffer (3 μg/ml, 100 μl/well) at 4° C. After washing with buffer A (PBS containing 0.1% Tween 20™), the plates were blocked with 3% BSA in PBS (250 μl/well) for 1 h at room temperature. Serum samples were diluted when necessary in buffer B (PBS containing 0.1% Tween 20™ and 1% BSA), and incubated overnight in the mAb coated plates at 4° C. The plates were washed and reacted with biotinylated rabbit anti-TR6 Ab (0.125 μg/ml in buffer B, 100 μl/well) at room temperature for 2 h. They were then washed and reacted with streptavidin-peroxidase (1:2,000 v/v in buffer B, Vector Laboratories, Burlingame, Calif.). After additional washes, a freshly prepared color development mixture (1:1 v/v mixture of tetramethyl benzidine solution and H₂O₂ solution, TMB Microwell Peroxidase Substrate System™, Kirkegard & Perry, Gaithersburg, Md.) was added to the plates (100 μl/well). The reaction was stopped after 20 min at room temperature with 0.1 N H₂SO₄ (100 μl/well), and OD₄₅₀ₙₘ was subsequently measured. Recombinant human TR as described previously (2) was used as standards. ELISA sensitivity was below 10 pg/ml.

### Immunohistochemistry (IHC)

Tumor tissues were fixed with formalin and embedded in paraffin. Sections 6 μm thick were mounted on glass slides pretreated with 0.1% poly-L-Lysine. They were then deparaffinized in xylene, dehydrated in graded ethanol, and soaked in 3% H₂O₂ for 10 min to eliminate endogenous peroxidase activity. Next the slides were submerged in citrate buffer (pH 6.0) and boiled at 92-98° C. in a microwave oven for 10 min. Subsequently, they were rinsed 3 times with PBS for 10 min each, and blocked with 10% normal goat serum in PBS for 1 h at room temperature. The slides were then reacted with the affinity-purified rabbit anti-TR6 Ab (1.67 μg/ml) at room temperature for 2 h. After washing, the slides were incubated with biotinylated goat-anti-rabbit antibody for 10 minutes. TR6 signal was revealed by streptavidin-peroxidase using DAB as a substrate according to instructions from the Histostain-Plus™ kit (Zymed Laboratories, South San Francisco, Calif.). TR6 signals were revealed in brown. Finally, the slides were counterstained with hematoxylin and sealed with Aqueous Mounting Media™ (Zymed). All the above described buffers and components are examples of reactants supporting the formation of a complex between TR6 and the ligand and the selective detection of this complex.

### Real time PCR

To measure TR6 gene copy number, DNA from fresh tumor samples was analyzed with real time PCR using Roche Lightcycler™. To detect TR6 gene signals, the upstream primer was 5′-CTTCTTCGCGCACGCTG-3′ (SEQ ID NO: 3), the downstream primer was 5′-ATCACGCCGGCACCAG-3′ (SEQ ID NO: 4), and the fluorogenic hybridization probe was 5′-FAM-ACACGATGCGTGCTCCMTCAGM-TAMARA (1). The samples were denatured at 95° C. for 30 sec, followed by 45 cycles of amplification (95° C., 0 sec; 50° C., 5 sec; 72° C., 50 sec), and the product was a 63-bp fragment. To detect β-globin gene signals as controls, the upstream primer was 5′-ACCCTTAGGCTGCTGGTGG (SEQ ID NO: 5), the downstream primer was 5′-GGAGTGGACAGATCCCCAAA (SEQ ID NO: 6), and the fluorogenic hybridization probe was 5′-CTACCCTTGGACCCAGAGGTTCTTTGAGTC-TAMARA-3′ (SEQ ID NO: 7) (12). The samples were denatured at 95° C. for 30 sec, followed by 45 cycles of amplification (95° C., 0 sec; 57° C., 5 sec; 72° C., 50 sec), and the product was a 71-bp fragment. TR6 and β-globin fluorescent signals were detected at 530 nm (F1) and 640 nm (F2), respectively, with a default setting of 1 for channel F1 and 15 for channel F2. The ratio of F1/F2 was calculated for each sample. A F1/F2 ratio falling within the range between the mean ±2 S.D. of the controls signifies 2 copies of the TR6 gene in the genome, with 99% confidence.

The present invention is illustrated in further details by the following non-limiting examples.

### EXAMPLE 1

### Assay for Detecting Tumor Patients Serum TR6 Levels and Comparing it with that of Test Sera Samples

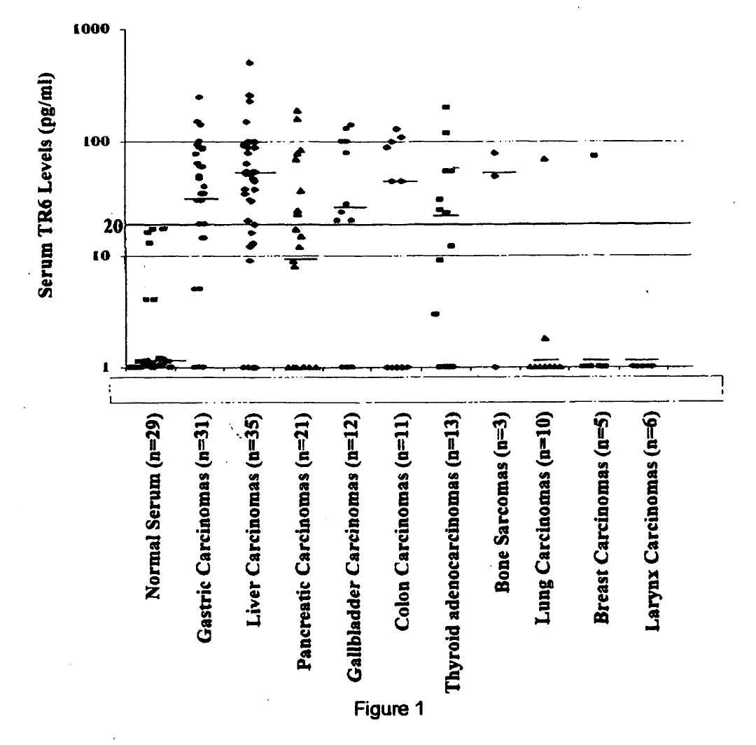

TR6 mRNA and protein are reportedly expressed at high levels in some lung, colon, gastric and esophageal tumors, and in malignant glioma cells (1,12,13). To explore diagnostic and prognostic applications based on TR6 expression, a sensitive ELISA to measure TR6 serum levels was established. As shown in FIG. 1, the sera from 29 healthy individuals and 146 tumor patients were tested for TR6. Most normal sera (15 out of 19) revealed TR6 levels below the ELISA sensitive range (10 pg/ml), and 4 samples were between 13 and 17 pg/ml. Therefore, an arbitrary positive threshold value of 20 pg/ml was set, above which a serum sample was considered TR6-positive. According to this criterion, 56.2% of all the tumor patients tested were TR6 positive (Table I below). The median serum TR6 level of all the tumor patients was 28 pg/ml, and the median serum TR6 level of all the serum TR6-positive patients was 55 pg/ml (Data not shown). Gastric, liver and gallbladder carcinoma patients had high TR6-positive incidences (70.7%, 74.3% and 75.9%, respectively). These rates were followed by those of colon carcinomas, thyroid adenocarcinomas and pancreatic carcinomas (54.5%, 53.8% and 38.1%, respectively). Lung adenocarcinomas disclosed quite a low incidence of TR6 (10.0%). The numbers of other tumor types were too small for meaningful comparison. These results also showed that TR6 serum levels could be elevated in certain carcinoma and sarcoma patients alike, and in gastrointestinal tumor patients as well as in patients with tumors of other origins (e.g., thyroid, bone, lung and breast).

TABLE I
Statistics of serum TR6 levels in tumour patients

| | | | Number of | % of patients |
|---|---|---|---|---|
| | | Median | patients with | with elevated |
| | Patient | serumTR6 | elevated TR6 | serum TR6 |
| | number | levels | levels | levels |
| Type of patients | (n) | (pg/ml) | (>20 pg/ml) | (>20/pg/ml) |
| Healthy Individuals | 29 | <10 | 0 | 0 |
| Gastric Carcinoma | 31 | 35 | 22 | 70.9 |
| Liver Carcinoma | 35 | 52 | 26 | 74.3 |
| Pancreatic | 21 | <10 | 8 | 38.1 |
| Carcinoma | | | | |
| Gallbladder | 12 | 28 | 9 | 75.9 |
| Carcinoma | | | | |
| Colon Carcinoma | 11 | 45 | 6 | 54.5 |
| Thyroid | 13 | 23 | 7 | 53.8 |
| Adenocarcinoma | | | | |
| Lung Carcinoma | 10 | <10 | 1 | 10.0 |
| Bone Sarcoma | 3 | 50 | 2 | 66.7 |
| Breast Carcinoma | 5 | <10 | 1 | 20 |
| Larynx Carcinoma | 5 | <10 | 0 | 0 |
| Total | 146 | | 82 | |

The statistical analysis showed that as a whole, the tumor patient sera had significantly higher levels of TR6 than control sera (Mann-Whitney rank sum test, p<0.001). Multiple comparisons between each different tumor type and the controls using Dunn's method showed that with the current sample size, patients of gastric, liver and gallbladder carcinomas had significantly higher levels of serum TR6 than the controls (Q=5.029, p<0.05 for gastric carcinomas; Q=5.664, p<0.05 for liver carcinomas; Q=3.4111, p<0.05 for gallbladder carcinomas). It is conceivable that if the sample size increases, the list of tumor types with statistically significantly higher serum TR6 levels will expand.

### EXAMPLE 2

### Assay for Distinguishing Patients having a Cell Proliferative Disease from Patients having an Infectious or Inflammatory Disease

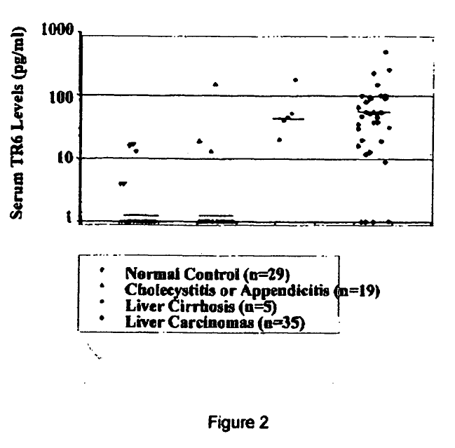

The serum TR6 levels of several tumor-related and tumor-unrelated conditions was measured (FIG. 2). In acute inflammatory diseases, such as cholecystitis or appendicitis, all but 1 of the sera tested appear TR6-negative. This indicates that serum TR6 levels are not generally affected by acute inflammation and infection. The serum with a high TR6 titer (150 ng/ml) was from a patient who had cholecystitis and a high fever. The reason for this exception was not clear, because neither high fever nor cholecystitis was correlated with elevated TR6 levels in the other patients of this group. Liver cirrhosis was found to be a condition with elevated serum TR6 levels according to five cases tested (all positive with a median of 45 pg/ml). As 74% of the serum TR6-positive liver carcinoma patients tested herein had liver cirrhosis, it is conceivable that some of their serum TR6 was derived from cirrhosis. Overall, it was found that almost all (98.8%) serum TR6-positive individuals (82 out of 83 cases) had malignancy, (the exception was a patient who had liver cirrhosis), and 97.9% healthy individuals and patients with acute infection (47 out of 48 cases) were serum TR6 negative.

### EXAMPLE 3

### Serum TR6 in Tumor Patients is Derived from Tumor Mass

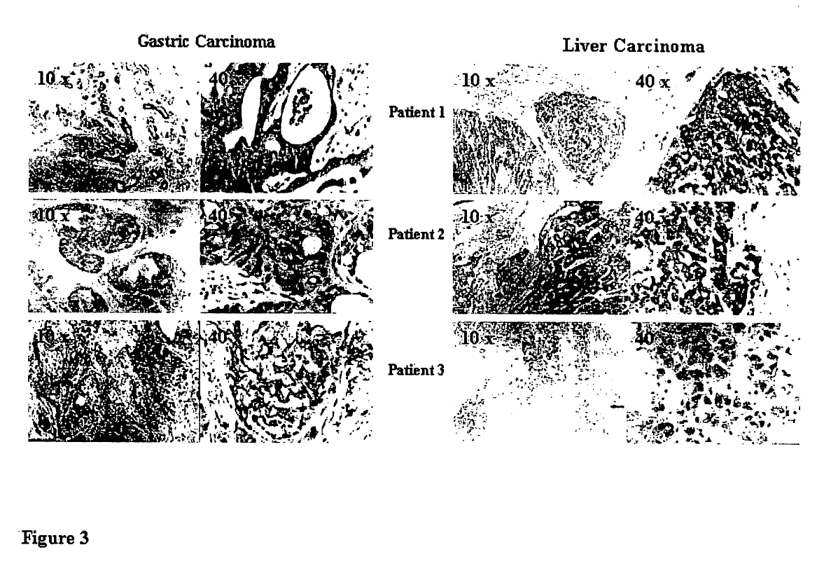

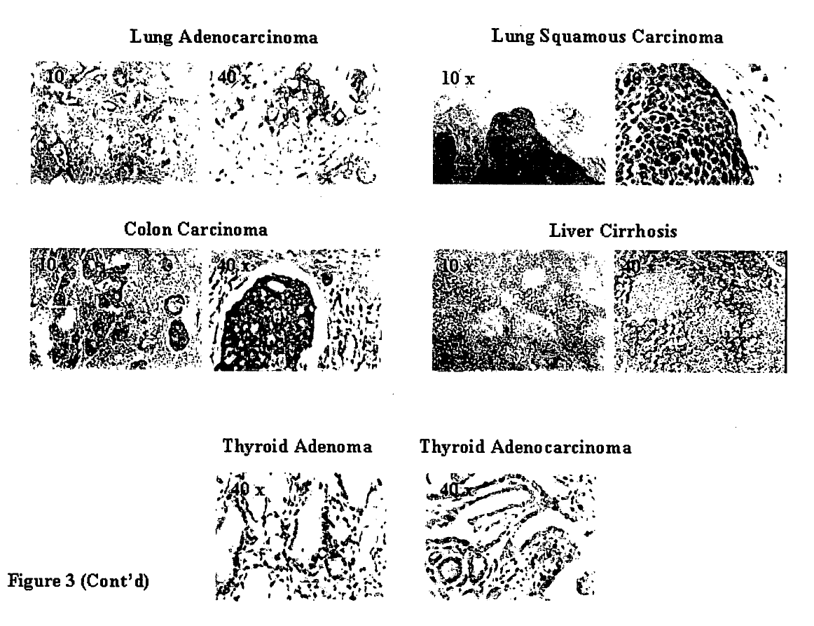

To identify the source of serum TR6, tumor samples were examined by IHC. TR6 signals were stained in brown. As shown in panels of FIG. 3, malignant cells in gastric carcinomas, colon carcinomas, liver carcinomas, lung adenocarcinimas and thyroid adenocarcinomas were strongly TR6-positive. The TR6 signal was restricted to cancer cell nests, and surrounding tissues were TR6 negative. Hepatocytes in liver cirrhosis contained moderately positive TR6 signals. On the other hand, thyroid adenoma was TR6-negative, and this was consistent with negative serum TR6 levels in these patients.

It is to be noted that not all tumors from TR6 serum positive patients were TR6 positive in IHC. In total, IHC analysis of 120 tumor samples from both serum TR6 positive and negative patients were carried out, and 22 samples were TR6 positive. The detection rate was only 18.0%, which was far lower than serum TR6 positive rate (56.2% as shown in Table I). The TR6 IHC positive incidence was clearly correlated with serum TR6 levels. Gastric carcinomas were used to demonstrate such correlation due to the large sample size available. As shown in Table II below, the patients were grouped according to their serum TR6 levels. Among the serum TR6 negative cases (0-20 pg/ml) and low serum TR6 cases (21-50 pg/ml), TR6 was not detectable with IHC (0/5 for the former group and 0/9 for the latter group). In the group with higher serum TR6 levels (51-150 pg/ml), TR6 could be detected in 2 out of 12 samples with IHC. For the cases with very high serum TR6 (>150 pg/ml), 4 out the 5 samples were TR6 positive with IHC. The Kendall's tau-b value of these data was 0.524, indicating highly significant correlation between serum TR6 levels and TR6 positive incidence in IHC (p<0.001). Among all the samples tested with ICH, there was not a case in which serum was negative while ICH was positive in TR6. Collectively, these results indicate that serum TR6 is a more sensitive parameter than TR6 detection with IHC.

TABLE II
Correlation between serum TR6 levels and IHC TR6 positive
incidence in gastric carcinomas

| TR6 level in | | TR6 Positive |
|---|---|---|
| serum(pg/ml) | Total Patient number (n) | Incidence in IHC |
| 0-20 | 5 | 0 |
| 21-50 | 9 | 0 |
| 51-150 | 12 | 2 |
| >150 | 5 | 4 |
| Total | 31 | 6 |

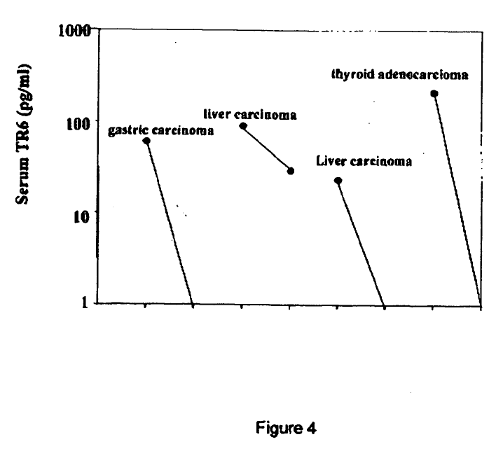

To ascertain that serum TR6 was mainly derived from tumor mass, sera from several patients before and after curative tumor resection were tested (FIG. 4). All the patients had high TR6 levels before surgery. Four to 6 weeks after the operations, only 1 patient had detectable serum TR6. The serum TR6 level of this liver carcinoma patient was decreased from 90 pg/ml before surgery to 29 pg/ml after the operation, but he also had liver cirrhosis, which was a condition associated with elevated serum TR6. These results indicate that tumor mass is the source of serum TR6, and even in the case of liver carcinoma accompanied by cirrhosis, a portion of serum TR6 is derived from the tumor mass. It should be noted that none of these 4 patients received chemotherapy after the surgery, and their decreased serum TR6 levels were not due to suppression of TR6 expression by drugs.

### EXAMPLE 4

### Serum TR6 Levels in Relation to Tumor Invasion and Metastasis

Thirty-one gastric cancer cases with standard UICC (Union International Contra la Cancer) clinical TNM (tumor node metastasis) classification (14) on the depth of tumor invasion (T1, T2, T3 and T4) and metastasis (N0, N1, N2 or N3 for tumor positive lymph nodes found in lymphadenectomy (proximal); M0 or M1 for detection of distant metastasis) were analyzed for their relationship with serum TR6 levels (Table III below). According to the Mann-Whitney non-parametric test, the (T1+T2) and (T3+T4) groups had no significant difference in their serum TR6 levels (p=0.202). On the other hand, the differences between (N2+N3) versus (N0+N1), and M1 versus M0 groups were significant (p=0.043 and 0.039, respectively). These analyses show that while the tumor metastasis is correlated with serum TR6 levels, the depth of tumor invasion into the gastric wall is not. Tumors were also grouped according to the overall TNM stage classification (i.e., ≦T2/N1/M0 versus >T2/N1/M0), and found that these two groups had significantly different serum TR6 levels (Mann-Whitney non parametric test, p<0.004). This indicates that a high serum TR6 level is an indicator of poor prognostic for gastric carcinomas.

TABLE III
Relationship between serum TR6 levels and TNM classification

| | | | Media | |
|---|---|---|---|---|
| | Patient | | serum | |
| Tumor invasion | number | SerumTR6 levels | TR6 levels | |
| and metastasis | (n) | (pg/ml) | (pg/ml) | P Value |
| T1-T2 | 15 | 0, 0, 0, 17, 24, 24, 30 | 30 | P = 0.202 |
| | | 54, 60, 60, 60, 100 | | |
| | | 110, 220, 250 | | |
| T3-T4 | 16 | 0, 19, 30, 35, 47 | 60 | |
| | | 47, 60, 60, 95, 140 | | |
| | | 150, 150, 190, 200 | | |
| | | 250, 270 | | |
| N0-N1 | 19 | 0, 0, 0, 0, 17, 19, 24 | 30 | P = 0.043 |
| | | 24, 30, 40, 47, 54, 60 | | |
| | | 60, 60, 110, 100, 200 | | |
| | | 270 | | |
| N2-N3 | 12 | 30, 35, 47, 60, 95, 95 | 95 | |
| | | 140, 150, 150, 190 | | |
| | | 220, 250 | | |
| M0 | 21 | 0, 0, 0, 0, 17, 19, 24 | 40 | P = 0.039 |
| | | 30, 35, 40, 47, 50, 60 | | |
| | | 60, 60, 60, 100, 110 | | |
| | | 200, 220, 270 | | |
| M1 | 10 | 30, 35, 47, 54, 95 | 95 | |
| | | 140, 150, 150, 190 | | |
| | | 250 | | |
| ≦T2/N1/M0 | 13 | 0, 0, 0, 0, 17, 24, 24 | 24 | P < 0.004 |
| | | 30, 40, 54, 60, 60 | | |
| | | 100 | | |
| ≧T2/N1/M0 | 18 | 19, 30, 35, 47, 47, 60 | 110 | |
| | | 60, 95, 110, 140, 150 | | |
| | | 50, 190, 200, 220 | | |
| | | 250, 250, 270 | | |

### EXAMPLE 5

### Serum TR6 Levels in Relation to Size

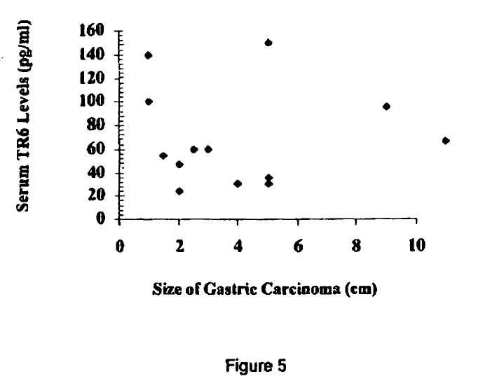

A possible correlation between tumor size and serum TR6 levels was investigated (FIG. 5). Twelve serum TR6 positive gastric carcinoma cases were analyzed. The correlation index (r) was 0.022063, and was less than r_{(n′10, 0.05)}=0.671. Thus, with this number of samples tested, gastric carcinoma sizes were not correlated to serum TR6 levels (p>0.05).

### EXAMPLE 6

### Serum TR6 Levels in Relation to Differentiation

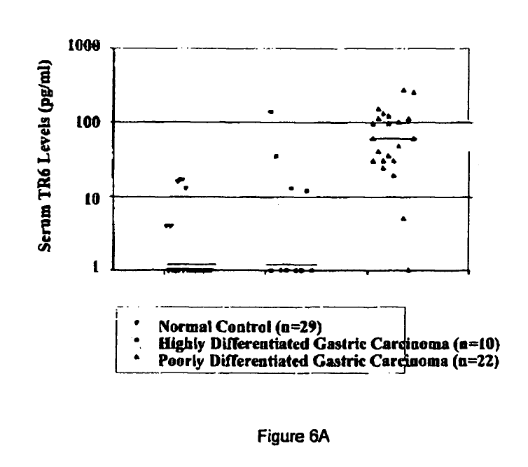

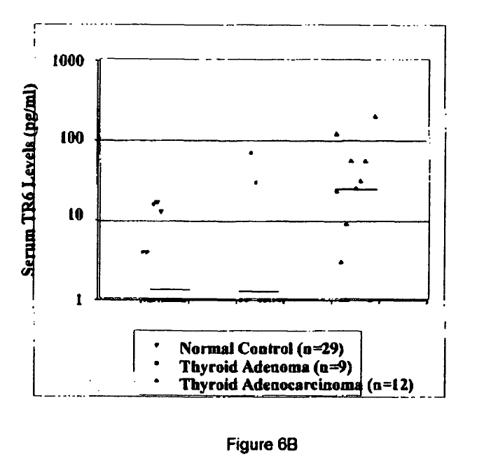

The tumor differentiation was examined for its correlation with serum TR6 levels. As seen in FIG. 6A, only 2 out of 10 (20%) highly differentiated gastric carcinoma sera were TR6-positive, compared with 20 of 22 (90.1%) sera from patients with poorly to intermediately differentiated gastric carcinomas. Similar analysis was conducted in thyroid adenomas versus thyroid carcinomas. In thyroid adenoma patients (FIG. 6B), only 2 out of 9 (22.2%) were TR6-positive, compared with 7 out of 12 (58.3%) in thyroid adenocarcinoma patients. According to Mann-Whitney non-parametric rank test, the TR6 levels were significantly different in highly and poorly differentiated gastric carcinomas (p<0.001). For thyroid adenomas versus adenocarcinomas, the p value was 0.064. It is conceivable that with a lager sample size, the difference would become significant. This demonstrates that a high serum TR6 level is predictive of poor prognosis, at least for gastric carcinomas, and this conclusion is consistent with that drawn from the TNM classification.

Examples 4 to 6 above demonstrate that TR6 concentration in blood, plasma or serum can be positively correlated with cell proliferative disease stage (through the presence of tumor invasion, proximal and distal metastasis and tumor differentiation.

### EXAMPLE 7

### Status of TR6 Gene Amplification in Different Tumors

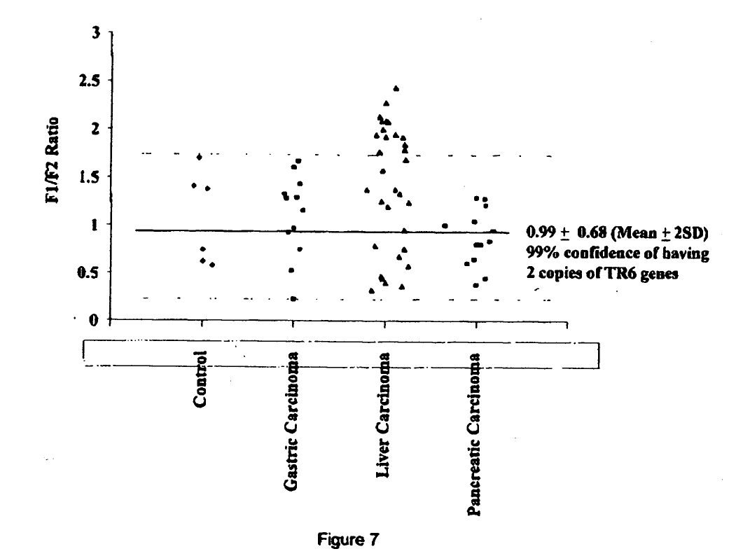

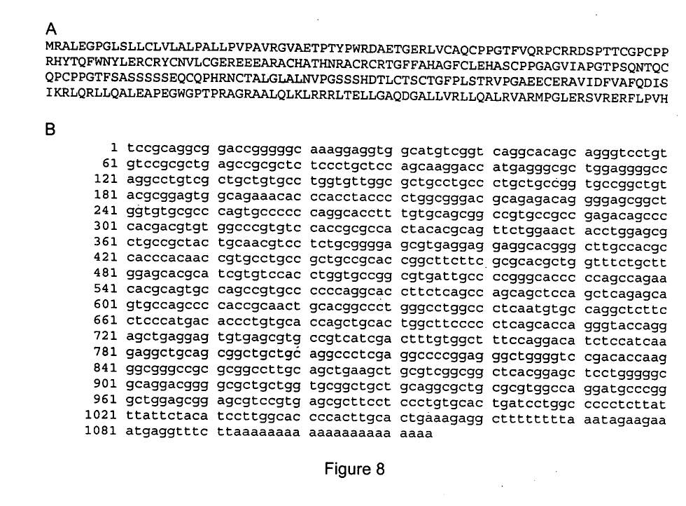

There are 2 reports concerning TR6 gene amplification in tumors. The initial report by Pitti et al. (1) showed that 8 of 18 lung tumors and 9 of 17 colon tumors had TR6 gene amplification. On the other hand, a subsequent report by Bai et al. stated that only 1 of 6 TR6 immunohistochemistry-positive gastrointestinal tumors (details of tumor types not described) had TR6 gene amplification (12). It was suspected that TR6 gene amplification might not be a universal phenomenon, but a feature of certain tumors. To pursue this line of enquiry, relative gene copy numbers of 12 gastric carcinomas, 31 liver carcinomas, and 16 pancreatic carcinomas were tested. DNA from the lymphocytes of 8 healthy donors was used as control. With real time PCR, ratios were calculated for fluorescent intensity (F1/F2) derived from the TR6 gene vs. an internal control, the β-globin gene (FIG. 7). According to normal controls and with 99% confidence, the range of the F1/F2 ratio signifying 2 copies of TR6 genes was between 0.32 and 1.67 (i.e., 0.99±0.68, mean ±2 SD). The range is illustrated between 2 dashed lines in FIG. 7, with the mean of 0.99 indicated by a solid line. According to this criterion, none of the 12 gastric carcinomas and 16 pancreatic carcinomas had TR6 gene amplification, while 15 of the 31 liver carcinomas (48.4%) had more that 2 copies of TR6 genes. Therefore, TR6 gene amplification is a feature in certain types of tumors, and elevated serum TR6 levels in most tumor patients are not due to TR6 gene amplification. TR6 gene amplification therefore does not appear to be a good marker.

### Discussion

It has been demonstrated hereinabove that a death decoy, DcR3/TR6, could be detected in the sera of tumor patients, and tumors were the source of serum TR6. Serum TR6 levels, taking gastric carcinomas as examples, were correlated with tumor TNM classification and degree of differentiation, and had prognostic values. Gene amplification was not the major mechanism of TR6 overexpression.

Although TR6 expression in tumor mass according to Northern, in situ hybridization or IHC has been reported previously (1,12, 15), the detection of TR6 in serum has special importance, because it offers a practical and easy-to-access method of for tumor diagnosis. In studying examples of the present invention, serum TR6 levels/concentrations were tested in healthy individuals, in patients with acute infection and inflammation, such as acute appendicitis or acute cholecystitis, in patients with liver cirrhosis, and in 146 patients with 10 different carcinomas and sarcomas. Excluding liver cirrhosis, serum TR6 above 20 pg/ml signified malignancy of some kind, with a false positive rate of 1.2% (1/83). Thus, the TR6 test could be conveniently included in the blood biochemistry of individuals undergoing routine health checks, or patients suspected to have tumors. An elevated TR6 serum level is an alarming signal for physicians and individuals concerned, and warrants further exhaustive examination and tests for malignancy, patients that have liver cirrhosis being excluded. Serum TR6 could also be a very useful parameter or marker for differential diagnosis between malignancies versus acute infection, because the latter was rarely serum TR6-positive (5.3%, i.e., 1/19). As the tumor mass is the source of serum TR6, serum TR6 could be monitored after curative tumor resection. Decrease below the threshold level of serum TR6 is an indication of successful tumor resection, and its re-appearance suggests tumor recurrence, tumor invasion or metastasis. It should be mentioned that patients with high serum TR6 levels remained serum TR6-positive at least for 1 week after tumor resection, and then converted to serum TR6-negative about 4 to 6 weeks after the surgery. This suggests that the half-life of endogenous TR6 is not very short.

The patients tested in examples presented herein were those who had undergone curative surgery, or at least endoscopic examination. Needless to say, this was a population already having visible tumors or other clinical indications of tumors. Serum TR6 could nevertheless be a useful parameter for diagnosis of early stage symptomless malignancies. For TR6 to be detected in serum, enough TR6 needs to be released from tumors, and this might be correlated with tumor size and/or TR6 secretion rates. The present invention shows that, in gastric carcinomas, serum TR6 was not correlated with tumor size, but rather closely with tumor differentiation status, which might be a key factor determining the rate of TR6 secretion. As shown in FIG. 5, 2 gastric carcinoma patients had tumor sizes of about 1 cm without metastasis, but their serum TR6 reached 100 pg/ml and 140 pg/ml, respectively. This suggests that positive serum TR6 levels might be detected when these tumors are much smaller than 1 cm in size. Therefore, for certain tumor types, serum TR6 could have an early diagnostic value.

More interestingly, the serum TR6 levels seem to have a prognostic value in gastric carcinomas, because TNM is a quite reliable prognostic parameter for gastric carcinomas (16) and serum TR6 levels were correlated with TNM stages, especially when the patients were grouped into <or =T2/N1/M0 versus >or =T3/N2/M1. It is reasonably predictable that this correlation also exists in other cell proliferative diseases. The correlation between TR6 serum levels and the N stages has especially useful clinical applications. While a more extensive lymphadenectomy is clearly beneficial for prolonged patient survival (17), it also brings about increased difficulty and risks in operation. Surgeons need to strike a balance between making unnecessarily extensive lymphadenectomy and leaving metastatic nodes untouched, and make such a decision based on visual inspection of the tumors during operation, because the degree of node metastasis (i.e., the N stage information) is only available from pathological examination after the surgery. As serum TR6 levels were correlated with the N stages, the knowledge of a high serum TR6 level before operation will allow surgeons to make an informed decision whether to perform D2⁺, D3 and/or D4 lymphadenectomy.

There are multiple serum tumor markers, such as prostate-specific antigen (18), CA125 (19), carcinoembryonic antigen (20), α-fetal protein (21), etc., currently in use for the diagnosis of tumors. These markers have no particular function in tumorigenesis. On the contrary, TR6 could well have an essential role in this regard. First, TR6 could bind to FasL, LIGHT and TL1A, and block Fas-, LTβR- and/or DR3-mediated tumor cell apoptosis (22), although experimental proof of the last mentioned is not yet available. Second, TR6 could interfere with binding between LIGHT and HveA/TR2, and thus repress the bidirectional costimulation mediated by this receptor-ligand pair (2,9,10). Third, Hsu et al. (11) reported an intriguing finding that soluble TR6 influences dendritic cell differentiation, which, in turn, drives T cells into the Th2 phenotype. If this happens in vivo, it might explain the low T-cell stimulatory capacity of dendritic cells isolated from tumors (23). Therefore, TR6 can protect tumor cells and repress the immune system through multiple pathways. It is conceivable that tumor cells gain a survival advantage by secreting TR6.

The next logical question is whether TR6 upregulation is a cause of tumors, or is simply an epiphenomenon, a result of linked gene replication, or enhanced transcription by yet-to-be-identified mechanisms, bearing no essential relevance to tumorigenesis. A definitive answer to this question will be obtained by assessing tumor incidences in TR6 transgenic animals. However, the available data from this study allows to make some reasonable assumptions. First of all, most of the tumors tested did not reveal gene amplification. This excludes the possibility that TR6 overexpression is purely a result of linked gene amplification, which occurs in many malignancies due to chaotic gene rearrangement and amplification. It also implies that TR6 overexpression is not necessarily only a consequence of malignancy. Indeed, it could be detected in liver cirrhosis, which is not a malignant condition. The liver cirrhosis of the patients presented herein was mostly the result of hepatitis B virus (HBV) infection, with 90% of these cases being HB-sAg- or HB-eAg- positive. These HBV-infected patients were at a very high risk (34.5 times higher than individuals without HBV infection) of having primary hepatomas (24-27). The correlation between high TR6 expression in HBV-evoked liver cirrhosis and the high risk of hepatomas in these HBV-infected patients suggests a plausible hypothesis concerning the role of TR6 in this type of primary hepatoma: because of a so-far-unknown mechanism(s), patients with liver cirrhosis/HBV infection have increased TR6 expression in their hepatocytes, which undergo rapid regenesis after some of them are damaged by the immune system due to HBV infection; by chance a few hepatocytes have tumorigenic mutations; TR6 prevents elimination of these mutant hepatocytes through mechanisms described above; the mutant hepatocytes gain survival advantage and develop into primary hepatomas. In this proposed scenario, TR6 is a facilitator of tumorigenesis. Further study is needed to prove this hypothesis, and to see whether it could be generalized for tumorigenesis of other types of TR6-expressing tumors.

It is obvious now that some tumors have amplified TR6 genes as initially reported by Pitti (1) and supported by recent publications (28,29). On the other hand, Bai et al. reported that tumors overexpress TR6 without gene amplification (12). It is to be noted that these findings are mostly based on studies on different types of tumors. In the present invention, TR6 gene amplification was only observed in some liver carcinomas but not in gastric and pancreatic carcinomas. It suggests that different types of tumors, or even the same types of tumors, might have different mechanisms to upregulate TR6 expression. This is certainly an interesting area for further studies. In liver carcinomas, chromosome regions harbouring some oncogenes (e.g. FGF3/FGF4, MET, MYC SAS/CDK4) at 1q22-24, 8p24 and 11q13 are frequently amplified (30). Since the TR6 gene is located at 20q13, its amplification in liver carcinomas is not due to co-amplification along with those oncogenes.

In view of the foregoing, serum TR6 is a novel parameter for tumor diagnosis, and prognosis.

As a whole, the tumor patient sera had significantly higher levels of TR6 than control sera (Mann-Whitney rank sum test, p<0.001). Multiple comparisons between each different tumor type and the controls using Dunn's method showed that gastric, liver and gallbladder carcinomas had significantly higher levels of serum TR6 than the controls (Q=5.029, p<0.05 for gastric carcinomas; Q=5.664, p<0.05 for liver carcinomas; Q=3.4111, p<0.05 for gallbladder carcinomas).

The Kendall's tau-b value of these data was 0.524, indicating highly significant correlation between serum TR6 levels and TR6 positive incidence in IHC (p<0.001) in gastric carcinomas.

Data from 31 gastric carcinoma patients were compiled and analyzed. According to the Mann-Whitney non-parametric test, the (T1+T2) and (T3+T4) groups had no significant difference in their serum TR6 levels (p=0.202). On the other hand, the differences between the (N2+N3) versus (N0+N1), and M1 versus M1 groups were significant (p=0.043 and 0.039, respectively). When the tumors were grouped according to the overall TNM stage classification (i.e., <or=T2/N1/M0 versus >T2/N1/M0), their serum TR6 levels were highly significantly different (p<0.004).

Although the present invention has been described hereinabove by way of preferred embodiments thereof, it can be modified, without departing from the spirit and nature of the subject invention as defined in the appended claims.

### References

1. Pitti, R. M., Marsters, S. A., Lawrence, D. A., Roy, M., Kischkel, F. C., Dowd, P., Huang, A., Donahue, C. J., Sherwood, S. W., Baldwin, D. T., Godowski, P. J., Wood, W. I., Gurney, A. L., Hillan, K. J., Cohen, R. L., Goddard, A. D., Botstein, D., and Ashkenazi, A. Genomic amplification of a decoy receptor for Fas ligand in lung and colon cancer. Nature, 396:699-703, 1998.

2. Zhang, J., Salcedo, T. W., Wan, X., Ullrich, S., Hu, B., Gregorio, T., Feng, P., Qi, S., Chen, H., Cho, Y. H., Li, Y., Moore, P. A., and Wu, J. Modulation of T-cell responses to alloantigens by TR6/DcR3. J.Clin.lnvest, 107: 1459-1468,2001.

3. Yu, K. Y., Kwon, B., Ni, J., Zhai, Y., Ebner, R., and Kwon, B. S. A newly identified member of tumor necrosis factor receptor superfamily (TR6) suppresses LIGHT-mediated apoptosis. J.Biol.Chem., 274:13733-13736, 1999.

4. Mauri, D. N., Ebner, R., Montgomery, R. I., Kochel, K. D., Cheung, T. C., Yu, G. L., Ruben, S., Murphy, M., Eisenberg, R. J., Cohen, G. H., Spear, P. G., and Ware, C. F. LIGHT, a new member of the TNF superfamily, and lymphotoxin alpha are ligands for herpesvirus entry mediator. Immunity., 8:21-30,1998.

5. Tamada, K., Shimozaki, K., Chapoval, A. I., Zhai, Y., Su, J., Chen, S. F., Hsieh, S. L., Nagata, S., Ni, J., and Chen, L. LIGHT, a TNF-like molecule, costimulates T cell proliferation and is required for dendritic cell-mediated allogeneic T cell response. J.lmmunol., 164:4105-4110, 2000.

6. Zhai, Y., Guo, R., Hsu, T. L., Yu, G. L., Ni, J., Kwon, B. S., Jiang, G. W., Lu, J., Tan, J., Ugustus, M., Carter, K., Rojas, L., Zhu, F., Lincoln, C., Endress, G., Xing, L., Wang, S., Oh, K. O., Gentz, R., Ruben, S., Lippman, M. E., Hsieh, S. L., and Yang, D. LIGHT, a novel ligand for lymphotoxin beta receptor and TR2/HVEM induces apoptosis and suppresses in vivo tumor formation via gene transfer. J.Clin.lnvest, 102:1142-1151, 1998.

7. Rooney, I. A., Butrovich, K. D., Glass, A. A., Borboroglu, S., Benedict, C. A., Whitbeck, J. C., Cohen, G. H., Eisenberg, R. J., and Ware, C. F. The lymphotoxin-beta receptor is necessary and sufficient for LIGHT—mediated apoptosis of tumor cells. J.Biol.Chem., 275:14307-14315, 2000.

8. Migone, T. S., Zhang, J., Luo, X., Zhuang, L., Chen, C., Hu, B., Hong, J. S., Perry, J. W., Chen, S. F., Zhou, J. X., Cho, Y. H., Ullrich, S., Kanakaraj, P., Carrell, J., Boyd, E., Olsen, H. S., Hu, G., Pukac, L., Liu, D., Ni, J., Kim, S., Gentz, R., Feng, P., Moore, P. A., Ruben, S. M., and Wei, P. TL1A is a TNF-like ligand for DR3 and TR6/DcR3 and functions as a T cell costimulator. Immunity., 16: 479-492, 2002.

9. Shi, G., Luo, H., Wan, X., Salcedo, T. W., Zhang, J., and Wu, J. Mouse T cells receive costimulatory signals from LIGHT, a TNF family member. Blood, 100:3279-3286, 2002.

10. Wan X, Zhang J, Luo H, Shi G, Kapnik E, Kim S, Kanakaraj P, Wu J. A TNF family member LIGHT transduces costimulatory signals into human T cells. J Immunol.169:6813-6821,2002.

11. Hsu, T. L., Chang, Y. C., Chen, S. J., Liu, Y. J., Chiu, A. W., Chio, C. C., Chen, L., and Hsieh, S. L. Modulation of dendritic cell differentiation and maturation by decoy receptor 3. J.lmmunol., 168:4846-4853, 2002.

12. Bai, C., Connolly, B., Metzker, M. L., Hilliard, C. A., Liu, X., Sandig, V., Soderman, A., Galloway, S. M., Liu, Q., Austin, C. P., and Caskey, C. T. Overexpression of M68/DcR3 in human gastrointestinal tract tumors independent of gene amplification and its location in a four-gene cluster. Proc.Natl.Acad.Sci.U.S.A, 97:1230-1235, 2000.

13. Roth, W., lsenmann, S., Nakamura, M., Platten, M., Wick, W., Kleihues, P., Bahr, M., Ohgaki, H., Ashkenazi, A., and Weller, M. Soluble decoy receptor 3 is expressed by malignant gliomas and suppresses CD95 ligand-induced apoptosis and chemotaxis. Cancer Res., 61:2759-2765, 2001.

14. Sobin L H, Wittekind C H. TNM classification of malignant tumor,5^{th} edition, International Union Against Cancer(UICC), New York, Wiley, 59-62, 1997.

15. Takahama Y, Yamada Y, Emoto K, Fujimoto H, Takayama T, Ueno M, Uchida H, Hirao S, Mizuno T, Nakajima Y. The prognostic significance of overexpression of the decoy receptor for Fas ligand (DcR3) in patients with gastric carcinomas. Gastric Cancer 5:61-68, 2002

16. D'Ugo D, Pacelli F, Persiani R, Pende V, lanni A, Papa V, Battista Doglietto G, Picciocchi A. Impact of the latest TNM classification for gastric cancer: retrospective analysis on 94 D2 gastrectomies. World J. Surg 26;6:672-7, 2002

17. Crookes P F. Gastric cancer. Clin Obstet Gynecol 45;3:892-903, 2002.

18. Stephenson, S. A., Verity, K., Ashworth, L. K., and Clements, J. A. Localization of a new prostate-specific antigen-related serine protease gene, KLK4, is evidence for an expanded human kallikrein gene family cluster on chromosome 19q13.3-13.4. J.Biol.Chem., 274:23210-23214,1999.

19. Yin, B. W. and Lloyd, K. O. Molecular cloning of the CA125 ovarian cancer antigen: identification as a new mucin, MUC16. J.Biol.Chem., 276: 27371-27375, 2001.

20. Thompson, J. A. Molecular cloning and expression of carcinoembryonic antigen gene family members. Tumour.Biol., 16; 1:10-16,1995.

21. Phillips, P., Rowland, R., Reid, D. P., and Coles, M. Alpha-fetoprotein in the diagnosis of hepatoma. Br.Med.J., 2:1432,1978.

22. Ashkenazi A. Targeting death and decoy receptors of the tumour-necrosis factor superfamily. Nat Rev Cancer. 2:420-30, 2002.

23. Banchereau, J., Briere, F., Caux, C., Davoust, J., Lebecque, S., Liu, Y. J., Pulendran, B., and Palucka, K. Immunobiology of dendritic cells. Annu.Rev.lmmunol., 18:767-811, 2000.

24. Yen, F. S. and Shen, K. N. Epidemiology and early diagnosis of primary liver cancer in China. Adv.Cancer Res., 47:297-329,1986.

25. Bosch, F. X., Ribes, J., and Borras, J. Epidemiology of primary liver cancer. Semin.Liver Dis., 19:271-285,1999.

26. Maddrey, W. C. Hepatitis B—an important public health issue. Clin.Lab, 47:51-55, 2001.

27. Brechot, C. Hepatitis B and C viruses and primary liver cancer. Baillieres Clin.Gastroenterol., 10:335-373,1996.

28. Ohshima K, Haraoka S, Sugihara M, Suzumiya J, Kawasaki C, Kanda M, Kikuchi M. Amplification and expression of a decoy receptor for fas ligand (DcR3) in virus (EBV or HTLV-I) associated lymphomas. Cancer Lett. 160:89, 2000.

29. Mild G, Bachmann F, Boulay J L, Glatz K, Laffer U, Lowy A, Metzger U, Reuter J, Terracciano L, Herrmann R, Rochlitz C. DCR3 locus is a predictive marker for 5-fluorouracil-based adjuvant chemotherapy in colorectal cancer. Int J. Cancer 102:254, 2002.

30. Takeo S, Arai H, Kusano N, Harada T, Furuya T, Kawauchi S, Oga A, Hirano T, Yoshida T, Okita K, Sasaki K. Examination of oncogene amplification by genomic DNA microarray in hepatocellular carcinomas: comparison with comparative genomic hybridization analysis. Cancer Genetics and Cytogenetics 130:127, 2001.

What is claimed is:

1. A method for diagnosing a cell proliferative disease expressing DcR3/TR6 in a patient, which comprises the step of measuring the concentration of DcR3/TR6 in the patient's blood, plasma or serum sample, wherein a concentration of DcR3/TR6 higher than that present in the serum of a patient not suffering of a proliferative disease expressing DcR3/TR6 is indicative of that patient suffering from said disease.

2. The method of claim 1, wherein said patient's sample comprises at least about 20 pg/mL DcR3/TR6.

3. The method of claim 1, wherein said disease is selected from the group consisting of gastric, liver, pancreatic, gallblader, colon, thyroid, lung, bone, larynx or breast cancers, and liver cirrhosis.

4. The method of claim 1, wherein the measuring step is selected from the group consisting of ELISA, radioimmunoassay, flow cytometry, fluorometry and immunoblotting.

5. The method of claim 1, wherein the detecting step comprises the use of an anti-DcR3/TR6 antibody.

6. The method of claim 1, wherein said patient is a patient who has undergone tumor resection at least about 4 weeks before said step of measuring DcR3/TR6 and wherein a concentration of DcR3/TR6 equal or lower than that present in the sample of a patient not suffering of a proliferative disease expressing DcR3/TR6 is indicative of the success of the tumor resection.

7. The method of claim 1, wherein said patient is one who has undergone tumor resection at least about 4 weeks before said step of measuring DcR3/TR6, and wherein a concentration of DcR3/TR6 higher than that present in the sample of a patient not suffering of a proliferative disease expressing DcR3/TR6 is indicative of a tumor recurrence.

8. The method of claim 1, wherein said patient is one who has undergone tumor resection at least about 4 weeks before said step of measuring DcR3/TR6 and whereby the tumor recurrence after tumor resection can be monitored.

9. The method of claim 1, wherein a concentration of DcR3/TR6 higher than that present in the sample of a patient not suffering from a proliferative disease expressing DcR3/TR6 is further indicative of the presence of metastasis or tumor invasion in the patient.

10. The method of claim 1, wherein said sample is serum.

11. A method for predicting whether a patient suffering from a cell proliferative disease is at risk of experiencing a disease progression or recurrence which comprises the step of measuring the concentration of DcR3/TR6 in the patient's blood, plasma or serum sample, wherein said sample DcR3/TR6 concentration positively correlates with the disease clinical stage.

12. The method of claim 11, wherein said patient's sample comprises at least about 20 pg/mL DcR3/TR6.

13. The method of claim 11, wherein said disease is selected from the group consisting of gastric, liver, pancreatic, gallblader, colon, thyroid, lung, bone, larynx or breast cancers, and liver cirrhosis.

14. The method of claim 11, wherein said disease is gastric cancer.

15. The method of claim 11, wherein the measuring step is selected from the group consisting of ELISA, radioimmunoassay, flow cytometry, fluorometry and immunoblotting.

16. The method of claim 11, wherein the detecting step comprises the use of an anti-DcR3/TR6 antibody.

17. The method of claim 11, wherein said sample is serum.

18. A composition of matter for the quantitative detection of DcR3/TR6 in a patient's blood, plasma or serum sample, which comprises a ligand to DcR3/TR6, reactants supporting the formation of a complex between said ligand and DcR3/TR6 and the detection of said complex, and a predetermined amount of DcR3/TR6 to be submitted to serial dilutions and to be mixed with a control sample, providing a DcR3/TR6 standard curve.

19. The composition of claim 18, wherein said ligand is an anti-DcR3/TR6 antibody.

20. The composition of claim 18, wherein said DcR3/TR6 serial dilutions achieve a concentration range expanding from 0.1 pg/mL to 1000 pg/mL sample.

21. A composition of matter for predicting a risk incurred by a patient suffering from a cell proliferative disease of experiencing a disease progression or recurrence, which comprises which comprises a ligand to DcR3/TR6, reactants supporting the formation of a complex between said ligand and DcR3/TR6 and the detection of said complex, and a predetermined amount of DcR3/TR6 to be submitted to serial dilutions and to be mixed with a control sample, providing a DcR3/TR6 standard curve.


SEQUENCE LISTING (8 sequences)

SEQ ID NO: 1 - DNA, 1114 bp, Homo sapiens
tccgcaggcggaccgggggcaaaggaggtggcatgtcggtcaggcacagcagggtcctgtgtccgcgctgagccgcgctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggcttttttttaaatagaagaaatgaggtttcttaaaaaaaaaaaaaaaaaaaaaa

SEQ ID NO: 2 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 3 - DNA, 17 bp, Artificial
  Oligonucleotide
cttcttcgcgcacgctg

SEQ ID NO: 4 - DNA, 16 bp, Artificial
  Oligonucleotide
atcacgccggcaccag

SEQ ID NO: 5 - DNA, 24 bp, Artificial
  Oligonucleotide
acacgatgcgtgctccaatcagaa

SEQ ID NO: 6 - DNA, 19 bp, Artificial
  Oligonucleotide
acccttaggctgctggtgg

SEQ ID NO: 7 - DNA, 20 bp, Artificial
  oligonucleotide
ggagtggacagatccccaaa

SEQ ID NO: 8 - DNA, 30 bp, Artificial
  oligonucleotide
ctacccttggacccagaggttctttgagtc